(19) United States
(12) Patent Application Publication
(10) Pub. No.: US 2014/0276560 A1
(43) Pub. Date: Sep. 18, 2014

(54) Infusion Management Platform For Medication Container Volume Tracking

(71) Applicant: CAREFUSION 303, INC., San Diego, CA (US)

(72) Inventors: Ritika Sehgal, San Diego, CA (US); Xiuchuan Pan, San Diego, CA (US); Brian Sullivan, San Diego, CA (US); Melissa Thompson, San Diego, CA (US)

(73) Assignee: CAREFUSION 303, INC., San Diego, CA (US)

(21) Appl. No.: 13/802,277

(22) Filed: Mar. 13, 2013

(51) Int. Cl. A61M 5/168; G06F 19/00

(52) U.S. Cl. CPC A61M 5/1684; G06F 19/3468
USPC 604/506; 604/151

(57) ABSTRACT

An infusion management platform specifies, for each of a plurality of containers, an identification for one of a plurality of patients, an identification for an infusion module, and attributes characterizing fluid within the container for infusion into the corresponding patient. Thereafter, the infusion management platform receives data from a plurality of infusion modules characterizing administration of fluid to each of a plurality of patients. It can then be determined by the infusion management platform for each container using a rule set, an amount of the fluid infused to the corresponding patient and an amount of fluid remaining in the container. Data can then be provided that characterizes the determined amount of infused fluid and the determined amount of remaining fluid for each container. Related apparatus, systems, techniques and articles are also described.

### TECHNICAL FIELD

The subject matter described herein relates to an infusion management platform for tracking volume of fluid/medication within infusion containers.

### BACKGROUND

In clinical settings, infusions for administration to a patient may be given by route of: intravenous (IV), subcutaneous, intra-arterial, epidural, enteral or irrigation of fluid spaces. These infusions may be delivered via a large volume pump, a syringe or by patient controlled analgesia. Infusions are often controlled by the hospital pharmacy and the pharmacy or prescriber can typically specify the volume of diluent that each infusion contains. However, the actual volume in the containers housing such infusions do not always correspond to the prescribed volumes such that there may be remaining medication/fluid after the prescribed volume is administered. In addition, in some cases, the amount of fluid initially provided within such containers (e.g., IV bags, etc.) can be overfilled or underfilled.

Further, caregivers often request repeated courses of treatment of the same medication/fluid. When there is a surplus of medication, it may be preferable to use any remaining amounts within a medication container. When there is not enough medication, new medication containers are required.

It is common practice for nurses who program the infusion at the patient's bedside to program the Volume To Be Infused (VTBI) for an amount less than the entire container volume. Nurses may program the infusion to be given in segments by programming multiple VTBIs until the entire container volume has been administered.

### SUMMARY

In one aspect, an infusion management platform specifies, for each of a plurality of containers, an identification for one of a plurality of patients, an identification for an infusion module, and attributes characterizing fluid within the container for infusion into the corresponding patient. Thereafter, the infusion management platform receives data from a plurality of infusion modules characterizing administration of fluid to each of a plurality of patients. The data can include the identification for the corresponding infusion module, the plurality of infusion modules being remote from the infusion management platform. It can then be determined by the infusion management platform for each container using a rule set, an amount of the fluid infused to the corresponding patient and an amount of fluid remaining in the container. Data can then be provided that characterizes the determined amount of infused fluid and the determined amount of remaining fluid for each container.

The provided data can include one or more of the following: data transmitted to a graphical user interface for displaying purposes, data transmitted to persistent storage, data transmitted to a remote computing system. At least one of the data providers is implemented by at least one data processor forming part of at least one computing system. At least one of the specifying, receiving, determining, and providing are implemented by at least one data processor forming part of at least one computing system.

The data can be used to determine one or more of the following: the maximum volume for each container, a cumulative infused volume, the start time of an infusion segment.

A maximum volume for the container can be equal to a nominal volume plus an overfill allowance. In addition, the provided data can include an estimated time at which the container will be empty.

A cumulative infused volume can be determined using the received data such that the determined amount of fluid remaining in the corresponding container is based on the corresponding determined cumulative infused volume. The cumulative infused volume can be equal to a summation of volume infused for each of at least one segment. Each segment can include a portion of the corresponding container programmed by the corresponding infusion module. Each segment can have a different order identification specifying an amount of the fluid prescribed for delivery to the corresponding patient. The received data can include messages indicating a start time for each segment.

It can be determined, based on the start time for each segment, whether a maximum container volume for the corresponding container has already been exceeded. The container can be characterized as being completed if the maximum volume has been exceeded. In addition, the infusion management platform can generate a new container that specifies the identification for the patient associated with the completed container, the identification for the infusion module that infused the fluid in the completed container, and attributes characterizing the fluid within the completed container.

Computer program products are also described that comprise non-transitory computer readable media storing instructions, which when executed one or more data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The subject matter described herein provides many advantages. For example, the current subject matter is advantageous in that it provides an infusion management platform that provides a view on all infusions being administered to a patient that takes into account overfill/underfill practices of a particular care facility.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a system diagram illustrating a computing landscape within a healthcare environment;

FIG. 2 is a first diagram illustrating a view of an infusion management platform;

FIG. 3 is a second diagram illustrating a view of the infusion management platform;

FIG. 4 is a third diagram illustrating a view of the infusion management platform;

FIG. 5 is a fourth diagram illustrating a view of the infusion management platform; and

FIG. 6 is a process flow diagram illustrating a method for implementation by an infusion management platform.

### DETAILED DESCRIPTION

FIG. 1 is a system diagram illustrating a computing landscape 100 within a healthcare environment such as a hospital. Various devices and systems, both local to the healthcare environment and remote from the healthcare environment, can interact via at least one computing network 105. This computing network 105 can provide any form or medium of digital communication connectivity (i.e., wired or wireless) amongst the various devices and systems. Examples of communication networks include a local area network (“LAN”), a wide area network (“WAN”), and the Internet. In some cases, one or more of the various devices and systems can interact directly via peer-to-peer coupling (either via a hardwired connection or via a wireless protocol such as Bluetooth or WiFi). In addition, in some variations, one or more of the devices and systems communicate via a cellular data network.

In particular, aspects of the computing landscape 100 can be implemented in a computing system that includes a back-end component (e.g., as a data server 110), or that includes a middleware component (e.g., an application server 115), or that includes a front-end component (e.g., a client computer 120 having a graphical user interface or a Web browser through which a user may interact with an implementation of the subject matter described herein), or any combination of such back-end, middleware, or front-end components. A client 120 and server 110, 115 are generally remote from each other and typically interact through the communications network 105. The relationship of the clients 120 and servers 110, 115 arises by virtue of computer programs running on the respective computers and having a client-server relationship to each other. Clients 120 can be any of a variety of computing platforms that include local applications for providing various functionality within the healthcare environment. Example clients 120 include, but are not limited to, desktop computers, laptop computers, tablets, and other computers with touch-screen interfaces. The local applications can be self-contained in that they do not require network connectivity and/or they can interact with one or more of the servers 110, 115 (e.g., a web browser).

A variety of applications can be executed on the various devices and systems within the computing landscape such as electronic health record applications, medical device monitoring, operation, and maintenance applications, scheduling applications, billing applications and the like.

The network 105 can be coupled to one or more data storage systems 125. The data storage systems 125 can include databases providing physical data storage within the healthcare environment or within a dedicated facility. In addition, or in the alternative, the data storage systems 125 can include cloud-based systems providing remote storage of data in, for example, a multi-tenant computing environment. The data storage systems 125 can also comprise non-transitory computer readable media.

Mobile communications devices (MCDs) 130 can also form part of the computing landscape 100. The MCDs 130 can communicate directly via the network 105 and/or they can communicate with the network 105 via an intermediate network such as a cellular data network. Various types of communication protocols can be used by the MCDs 130 including, for example, messaging protocols such as SMS and MMS.

Various types of medical devices 140 can be used as part of the computing landscape 100. For example, the landscape can include comprise various systems/units for delivering fluid (including medication) to a patient. On particular type of medical device 140 is an infusion module 140A. The infusion modules 140A can include various types of infusion pumps including peristaltic infusion pumps, large volume infusion pumps, and syringe pumps. The infusion modules 140A can be directly coupled to the network 105 and/or they can be coupled to a medical device 140 which is, in turn, coupled to the network 140.

The medical devices 140 can comprise, unless otherwise specified, any type of device or system with a communications interface that characterizes one or more physiological measurements of a patient and/or that characterize treatment of a patient. In some cases, the medical devices 140 communicate via peer to peer wired or wireless communications with another medical device 140 (as opposed to communicating with the network 105). For example, the medical device 140 can comprise a bedside vital signs monitor that is connected to other medical devices 140, namely a wireless pulse oximeter and to a wired blood pressure monitor. One or more operational parameters of the medical devices 140 can be locally controlled by a clinician, controlled via a clinician via the network 105, and/or they can be controlled by one or more of a server 115, 120, a client 125, a MCD 130, and/or another medical device 140.

The computing landscape 100 can provide various types of functionality as may be required within a healthcare environment such as a hospital. For the medical devices 140 can provide data characterizing one or more physiological measurements of a patient and/or treatment of a patient (e.g., medical device 140 can be an infusion management system, etc.). The data generated by the medical devices 140 can be communicated to other medical devices 140, the servers 110, 115, the clients 120, the MCDs 130, and/or stored in the data storage systems 125.

The computing landscape 100 can also include at least one medication ordering system 145. The medication ordering system 145 is coupled to the network and enables orders (e.g., prescriptions, etc.) to be generated and monitored. The medication order system 145 can be accessed, for example, via the one of the clients 120 and MCDs 130 via the application server 115. The medication ordering system 145 can specify a plurality of medications and/or other fluids to be infused into a patient over a pre-defined period of time and according to a pre-defined sequence via at least one infusion module 140A. This orders can be stored in the data storage 125 and/or pushed out to other clients 120, an MCD 130, and/or one or more of the medical devices 140. In some cases, caregivers alter the timing and sequence of such medication delivery based on reactions from the patient (as measured by various physiological sensors, etc.).

One more of the medical devices 140 (such as infusion modules 140A) can monitor an amount of fluid (e.g., medication, etc.) delivered to a patient. Fluids delivered to patients are referred to herein as infusions. Unless otherwise specified, references herein to medications should also be construed to include non-medication fluids (e.g., blood, saline, etc.) for delivery to a patient via an infusion module 140A.

As noted above, containers housing fluids such as medication often vary from the volumes ordered by a pharmacist/prescriber. A software-implemented infusion management platform 150 can be provided that includes a graphical user interface for tracking and monitoring infusions for one or more patients. The infusion management platform 150 communicates with the infusion modules 140A via the network 105. The infusion modules 140A can directly or indirectly provide various attributes relating to a particular infusion to the infusion management platform 150 (e.g., patient identifier, medication container identifier, medication type, rate of medication administration, infusion module identifier, etc.). Such attributes can be provided, for example, via messages sent from the infusion modules 140A. In some cases, the infusion management platform 150 receives medication orders from the medication ordering system 145 and then associates such orders with particular infusion modules 140A and/or particular patients (who are later associated with the infusion modules 140A).

FIG. 2 is a view 200 illustrating a sample graphical user interface of the software infusion management platform. The graphical user interface provides various graphical user interface elements and displays data that enables a view of multiple concurrent infusions for one or more patients. In this example, data regarding a series of infusions (each illustrated in a separate row) provided to at least one patient are displayed. Such data can be periodically or dynamically updated based on data received from the infusion modules 140A. A first column 205 can list a patient identifier (which differs from the patient's name). A second column 210 can identify a particular type of infusion (e.g., continuous, intermittent, fluid, etc.). A third column 215 can list the name of the patient. A fourth column 220 can identify the fluid being infused and/or the infusion treatment. A fifth column 225 can identify a dosage level for the infusion. A sixth column 230 can identify a flow rate for the infusion. A sixth column 235 can list an estimated time at which the container housing the infused fluid will be empty. A seventh column 240 provides an estimated volume remaining in the container housing the infused fluid. An eighth column 245 lists a current status of the infusion. A ninth column 250 lists when the data regarding the particular infusion was last updated (in cases in which the data is not continually updated). A tenth column 255 lists a priority associated with the infusion. It will be appreciated that the particular view of diagram 200 is for illustration purposes only and that additional and/or less information characterizing ongoing infusions may be displayed depending on the desired implementation. In addition, various graphical user interface elements can be provided to enable a user to have different views of the data being displayed and/or to otherwise manipulate or consume the displayed data.

The infusion management platform 150 can calculate (using, for example, one or more pre-defined rules) concentrations for medications as well as volumes of containers housing such medications in a variety of manners (which in turn is used to calculate the estimated volume remaining as illustrated in the seventh column 240). In one example, medication concentration can be defined as a ratio of the drug units to a diluent volume (e.g., CEFTAZidime 1000 mg/50 ml). For most medication containers the diluent volume is the container's nominal volume. This rule does not apply for intermittent or continuous medications that have concentrations defined per unit volume (medication amount per 1 mL, for example Cefazolin 5 mg/1 ml). By tracking the diluent volume, one can compute the container volume. Container Nominal Volume=Diluent Volume.

The infusion management platform 150 can account for variations in volumes of containers. In most cases, the actual volume of a container is greater than the container's nominal volume (based on hospital's practices to overfill the container). Individual hospital practices can standardize the overfill (volume) percentage for their pharmacies. Maximum volume for can be computed as: Maximum Container Volume=Container Nominal Volume*(1+overfill %).

In some cases, infusions may be programmed with a diluent volume of 1 mL. In these scenarios, the volume to be infused (VTBI) for the initial infusion can be used as the container maximum volume, in which case: Maximum Container Volume=VTBI.

To track an infusion container, the infusion management platform 150 can maintain the Maximum Container Volume and compare against the cumulative infused volume of each container. The infusion module 140A can provide the volume infused as a part of the infusion message (i.e., the data transmitted from the corresponding infusion module 140A). The cumulative infused volume can be equal to the summation of infused volume of all segments that can be identified to a container. A segment as used herein can be defined as a portion of the container that is programmed and administered at a given time and is identified with a new order identification. Such an order can originate, for example, at the medication ordering system 145 (e.g., a pharmacy and/or EMR system, etc.). Cumulative Volume Infused=Summation of Volume Infused for all Segments.

The infusion management platform 150 can maintain a cumulative sum of the volume infused for a container. The infusion management platform 150 can identify and create a container with a new order ID associated with an infusion module 140A. The container information can then be maintained by the infusion management platform 150.

When the infusion management platform 150 receives a message that indicates a segment start (START or RESTART message with an order ID—assigned at the programming of an infusion), an algorithm can be triggered to identify if the infusion relates to a container that the infusion management platform 150 has already stored. If the algorithm determines that the Maximum Container Volume has been infused and or exceeded, the existing container and its associated infusion is considered to have been completed and a new one created.

For each new container identified, the infusion management platform 150 can store associated infusion values for the infusion including, for example, one or more of: patient ID, infusion module ID, drug: name, concentration (drug amount/diluent volume) and national drug code (NDC), where applicable, and infusion type: primary or secondary. These values define a unique container. If the list of containers, being maintained by the infusion management platform 150, does not match the infusion details (above), the algorithm can create a new infusion container (the current container is considered complete). In some implementations, when considering the Cumulative Volume Infused the algorithm can account for primary or secondary infusions/containers.

The infusion management platform 150 can use the Cumulative Volume Infused of a Container and the Volume to Be Infused (VTBI) in an infusion message received from an infusion module 140A to identify if it is the same container or a new one. If the summation of the Cumulative Volume Infused and VTBI exceeds the Maximum Container Volume of this container, the algorithm can mark the previous container as completed and a new container can be created. Otherwise, the algorithm can determine that the infusion messages are a new segment of an existing container.

So, for a new segment received by the infusion management platform 150, if the Cumulative Volume Infused+VTBI<=Maximum Container Volume, then the segment can be identified being part of an existing/current container. Otherwise, it can be considered that a new container is being used (i.e. Cumulative Volume Infused+VTBI>Maximum Container Volume).

FIGS. 3-5 are diagrams 300, 400, 500 illustrating different views of the infusion management platform that illustrate data regarding drug amount/diluent volume 305, cumulative volume infused 310, and volume to be infused 315. With reference to diagram 300 of FIG. 3, a medication bag of 50 ml of CEFTAZidime can be coupled to the patient, and the caregiver (e.g., nurse, etc.) programs that 30 ml of the medication is to be infused to the patient (as indicated by the VTBI column 315). With reference to diagram 400 of FIG. 4, after the 30 ml of medication has been infused into the patient, the caregiver programs another 20.5 ml of the medication (as indicated by the VTBI column 315). Although 50.5 ml exceeds the bag volume 50 ml, the variation falls within a pre-defined range of tolerance. With reference to diagram 500 of FIG. 5, after the second infusion of 20.5 ml is finished, the caregiver programs an infusion of an additional 5 ml (as indicated by the VTBI column 315). As this additional amount falls outside the range of tolerance, the infusion management platform associates this additional infusion as being a new container.

FIG. 6 is a process flow diagram 600 illustrating a method in which, at 610, an infusion management platform specifies, for each of a plurality of containers, an identification for one of a plurality of patients, an identification for an infusion module, and attributes characterizing fluid within the container for delivery to the corresponding patient. Thereafter, at 620, the infusion management platform receives data from a plurality of remote infusion modules that characterizes administration of fluid to each of a plurality of patients and that includes the identification for the corresponding infusion module. Subsequently, at 630, the infusion management platform determines, for each container, an amount of the fluid infused to the corresponding patient and an amount of fluid remaining in the container. Such determination can be made, for example, using a rule set that defines how such calculations can be generated. Once this determination has been made, at 640, data characterizing the determined amount of infused fluid and the determined amount of remaining fluid for each container is provided (e.g., displayed, stored, loaded, transmitted, etc.).

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g., mouse, touch screen, etc.), and at least one output device.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow(s) depicted in the accompanying figures and/or described herein do not necessarily require the particular order shown, or sequential order, to achieve desirable results. Other implementations may be within the scope of the following claims.

What is claimed is:

1. A computer-implemented method:
specifying, by an infusion management platform for each of a plurality of containers, an identification for one of a plurality of patients, an identification for an infusion module, and attributes characterizing fluid within the container for infusion into the corresponding patient;
receiving, by the infusion management platform, data from a plurality of infusion modules characterizing administration of fluid to each of a plurality of patients, the data comprising the identification for the corresponding infusion module, the plurality of infusion modules being remote from the infusion management platform;
determining, by the infusion management platform for each container using a rule set, an amount of the fluid infused to the corresponding patient and an amount of fluid remaining in the container; and
providing data characterizing the determined amount of infused fluid and the determined amount of remaining fluid for each container.

2. A method as in claim 1, wherein providing data comprises at least one of: displaying the data in a graphical user interface, loading the data, storing the data, and transmitting the data to a remote computing system.

3. A method as in claim 1, wherein at least one of the specifying, receiving, determining, and providing are implemented by at least one data processor forming part of at least one computing system.

4. A method as in claim 1, wherein the determining comprises:
determining a maximum volume for each container.

5. A method as in claim 4, further comprising:
determining, using the received data, a cumulative infused volume for each container;
wherein the determined amount of fluid remaining in the corresponding container is based on the corresponding determined cumulative infused volume.

6. A method as in claim 5, wherein cumulative infused volume is equal to a summation of volume infused for each of at least one segment, wherein each segment comprises a portion of the corresponding container programmed by the corresponding infusion module.

7. A method as in claim 6, wherein each segment has a different order identification specifying an amount of the fluid prescribed for delivery to the corresponding patient.

8. A method as in claim 7, wherein the received data comprises messages indicating a start time for each segment.

9. A method as in claim 8, further comprising:
determining, based on the start time for each segment, whether a maximum container volume for the corresponding container has already been exceeded;
characterizing the container as being completed if the maximum volume has been exceeded; and
generating, by the infusion management platform, a new container that specifies the identification for the patient associated with the completed container, the identification for the infusion module that infused the fluid in the completed container, and attributes characterizing the fluid within the completed container.

10. A method as in claim 1, wherein a maximum volume for the container is equal to a nominal volume plus an overfill allowance.

11. A method as in claim 1, wherein the provided data comprises an estimated time at which the container will be empty.

12. A non-transitory computer program product storing instructions, which when executed by at least one data processor of at least one computing system, result in operations comprising:
specifying, by an infusion management platform for each of a plurality of containers, an identification for one of a plurality of patients, an identification for an infusion module, and attributes characterizing fluid within the container for infusion into the corresponding patient;
receiving, by the infusion management platform, data from a plurality of infusion modules characterizing administration of fluid to each of a plurality of patients, the data comprising the identification for the corresponding infusion module, the plurality of infusion modules being remote from the infusion management platform;
determining, by the infusion management platform for each container using a rule set, an amount of the fluid infused to the corresponding patient and an amount of fluid remaining in the container; and
providing data characterizing the determined amount of infused fluid and the determined amount of remaining fluid for each container.

13. A computer program product as in claim 12, wherein providing data comprises at least one of: displaying the data in a graphical user interface, loading the data, storing the data, and transmitting the data to a remote computing system.

14. A computer program product as in claim 12, wherein the determining comprises:
determining a maximum volume for each container.

15. A computer program product as in claim 14, further comprising:
determining, using the received data, a cumulative infused volume for each container;
wherein the determined amount of fluid remaining in the corresponding container is based on the corresponding determined cumulative infused volume.

16. A computer program product as in claim 15, wherein cumulative infused volume is equal to a summation of volume infused for each of at least one segment, wherein each segment comprises a portion of the corresponding container programmed by the corresponding infusion module;
wherein each segment has a different order identification specifying an amount of the fluid prescribed for delivery to the corresponding patient;
wherein the received data comprises messages indicating a start time for each segment.

17. A computer program product as in claim 16, wherein the operations further comprise:
determining, based on the start time for each segment, whether a maximum container volume for the corresponding container has already been exceeded;
characterizing the container as being completed if the maximum volume has been exceeded; and
generating, by the infusion management platform, a new container that specifies the identification for the patient associated with the completed container, the identification for the infusion module that infused the fluid in the completed container, and attributes characterizing the fluid within the completed container.

18. A computer program product as in claim 12, wherein a maximum volume for the container is equal to a nominal volume plus an overfill allowance.

19. A computer program product as in claim 12, wherein the provided data comprises an estimated time at which the container will be empty.

20. A system comprising:
at least one data processor; and
memory storing instructions, which when executed by the at least one data processor, result in operations comprising:
specifying, by an infusion management platform for each of a plurality of containers, an identification for one of a plurality of patients, an identification for an infusion module, and attributes characterizing fluid within the container for infusion into the corresponding patient;
receiving, by the infusion management platform, data from a plurality of infusion modules characterizing administration of fluid to each of a plurality of patients, the data comprising the identification for the corresponding infusion module, the plurality of infusion modules being remote from the infusion management platform;
determining, by the infusion management platform for each container using a rule set, an amount of the fluid infused to the corresponding patient and an amount of fluid remaining in the container; and
providing data characterizing the determined amount of infused fluid and the determined amount of remaining fluid for each container.
